(19) United States
(12) Patent Application Publication
(10) Pub. No.: US 2019/0240851 A1
(43) Pub. Date: Aug. 8, 2019

(54) BEDSIDE MEDICAL SCISSORS

(71) Applicant: Paul Castaneda, Santa Rosa, CA (US)

(72) Inventor: Paul Castaneda, Santa Rosa, CA (US)

(21) Appl. No.: 15/891,673

(22) Filed: Feb. 8, 2018

(51) Int. Cl. B26B 13/12; B26B 13/22

(52) U.S. Cl. CPC B26B 13/12; A61B 17/3201; B26B 13/22

(57) ABSTRACT

Improved bedside medical scissors are provided, which include a first scissor component including a first handle component and a second scissor component including a second handle component. The first scissor component includes a plurality of circles, each with different sized diameters, and the second scissor component includes a plurality of measurement increments. The improved bedside medical scissors also feature a removable light positioned in the first handle component. The removable light being operable while positioned in the first handle component and while removed from the bedside medical scissors.

### CROSS-REFERENCE TO RELATED APPLICATIONS

N/A

### BACKGROUND OF THE INVENTION

### 1. Field of the Invention

The present invention generally relates to scissors, but more particularly to bedside medical scissors.

### 2. Description of Related Art

Medical utility scissors, or shears, are often used by bedside nurses while caring for patients. These scissors are useful for cutting small items such as tape and gauze. However, there are many limitations and disadvantages of the current medical utility scissors of the prior art. Further, often nurses need multiple tools and scissors for their required shift, which can be burdensome. Consequently, there is a need for a single pair of scissors designed for the specific needs while taking care of inpatients in a hospital.

### BRIEF SUMMARY OF THE INVENTION

In one embodiment of the present invention bedside medical scissors are provided, comprising a first scissor component including a first handle component, wherein the first handle component is formed to include a first hole configured to receive a thumb of a user; a second scissor component including a second handle component, wherein the second handle component is formed to include a second hole configured to receive at least one finger of the user; the first scissor component and the second scissor component each including a blade, wherein the first scissor component and the second scissor component are attached and configured to pivot via a pivot element forming a shear; and a removable light positioned in the first handle component, wherein the removable light is operable while positioned in the first handle component and operable while removed from the first handle component of the bedside medical scissors.

In one embodiment, the first scissor component includes a plurality of circles, each circle of the plurality of circles having a different sized diameter. In one embodiment, the plurality of circles range from two to six millimeters in diameter. In another embodiment, the second scissor component includes a plurality of measurement increments. In one embodiment, the plurality of measurement increments is from zero to five centimeters, and the plurality of measurement increments are located above the blade. In yet another embodiment, the first scissor component includes a first section of clamp and the second scissor component includes a second section of clamp, such that a clamp is provided. In one embodiment, the clamp is configured to attach to intravenous (IV) lines. In one embodiment, the bedside medical scissors are constructed of various materials which are configured to be autoclaved when the removable light is removed from the first handle component. In another embodiment, the removable light includes a micro USB port configured to enable the charging of a rechargeable battery. In one embodiment, the removable light includes a LED light powered by the rechargeable battery. In yet another embodiment, the removable light includes a connecting loop enabling the removable light to be attached to a lanyard. In one embodiment, a pocket clip positioned on the second handle component is provided.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a perspective view of bedside medical scissors according to an embodiment of the present invention.

FIG. 2 is a front view of the bedside medical scissors according to an embodiment of the present invention.

FIG. 3 is a back view of the bedside medical scissors according to an embodiment of the present invention.

FIG. 4 is a front view of the bedside medical scissors in an open position according to an embodiment of the present invention.

FIG. 5 is a perspective view of the removable light removed from the bedside medical scissors according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide bedside medical scissors. The term “scissors” can also be as referred to as “a pair of scissors”.

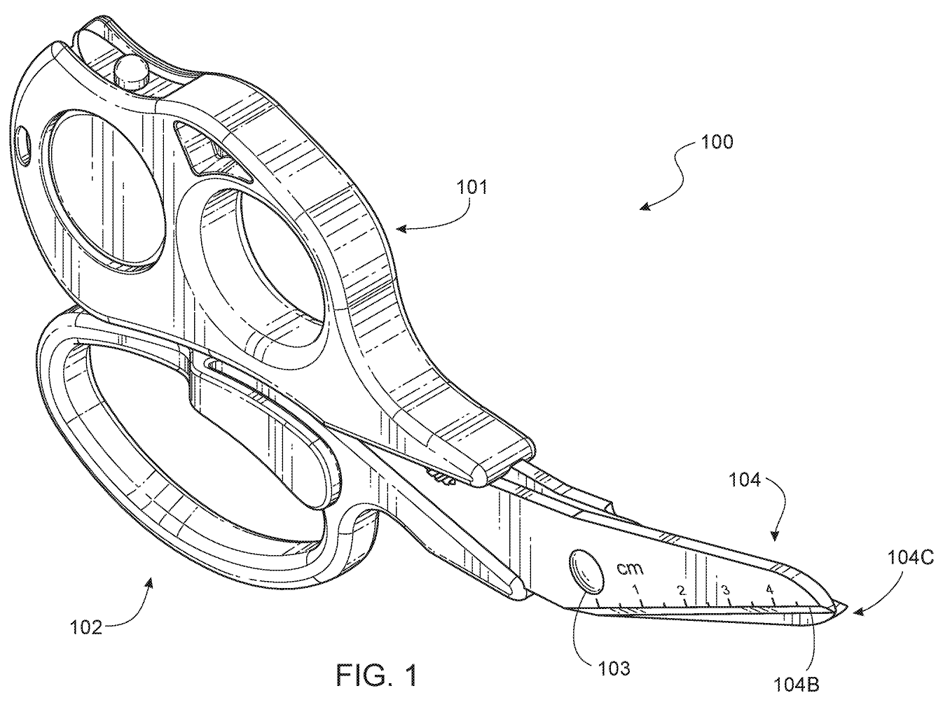

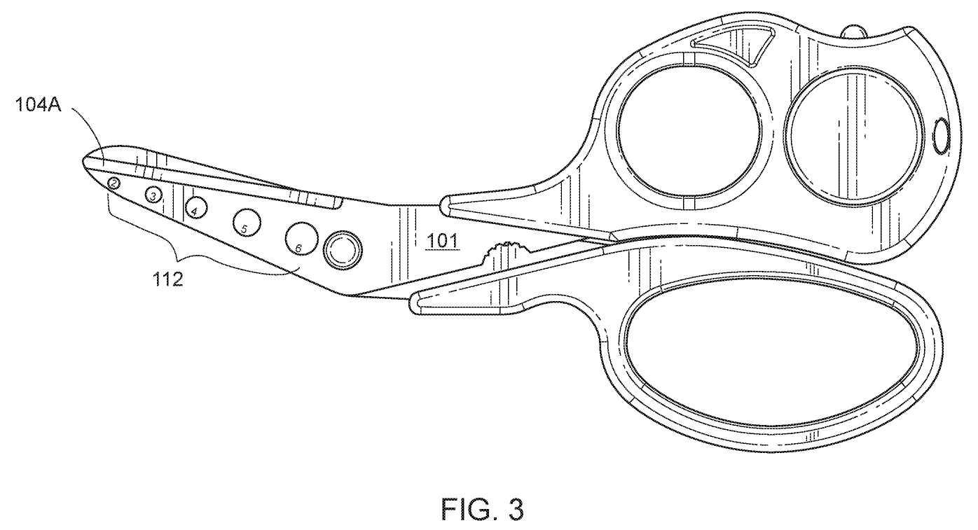

FIG. 1 is a perspective view of bedside medical scissors according to an embodiment of the present invention. Referring to FIG. 1, the bedside medical scissors 100 are illustrated. The scissors comprise a first scissor component 101 and a second scissor component 102 attached and pivoted via element 103. In one embodiment, each of the first and second scissor components includes blades 104A (FIG. 3) and 104B forming a shear 104. In one embodiment, the shear is used to cut bandages, dressings, name bands, tape, clothing, etc. In one embodiment, the tips 104C of each blade at the shear are blunt as to not gouge a patient's skin during cutting. In one embodiment, the bedside medical scissors, and more specifically, the first and second scissor components are constructed from stainless steel.

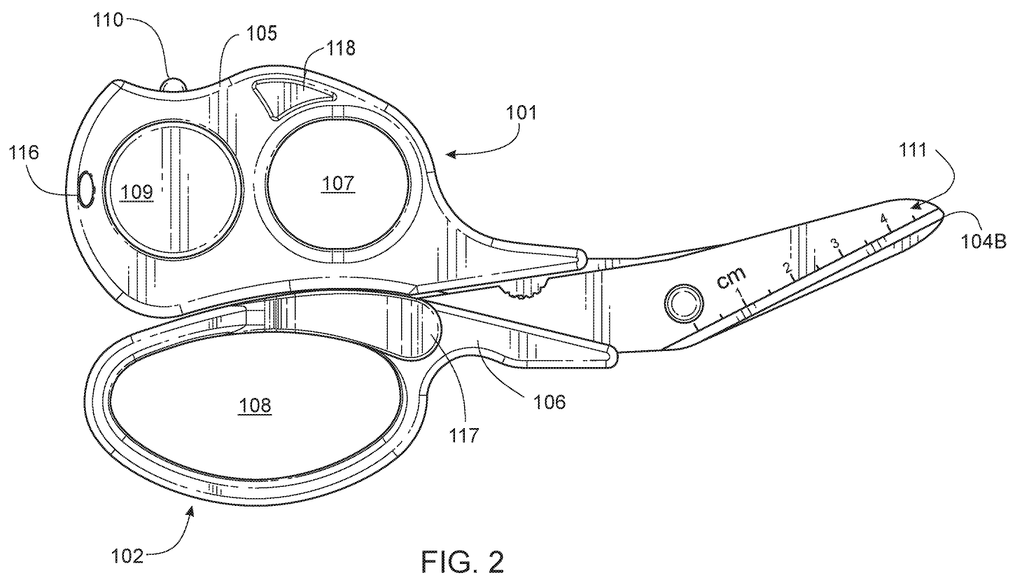

FIG. 2 is a front view of the bedside medical scissors according to an embodiment of the present invention. Referring now to FIG. 2, the first scissor component includes a first handle component 105 and the second scissor component includes a second handle component 106. In one embodiment, the first handle component is formed to include a first hole 107 configured to receive a thumb of a user. In one embodiment, the second handle component is formed to include a second hole 108 configured to receive the user's fingers. It is a user's preference how many figures are inserted into the second hole. It is a particular advantage of the present invention, that the first and second hole that may be utilized by users having smaller hands. However, a user with larger hands can still operate the bedside medical scissors effectively and comfortably. In one embodiment, the first and second handle components are constructed from polyethylene. However, it should be understood that the material can vary without departing from the spirit and scope of the invention. The bedside medical scissors can be modified specifically for left and right handed users; however the design illustrated is intended primarily for right handed users.

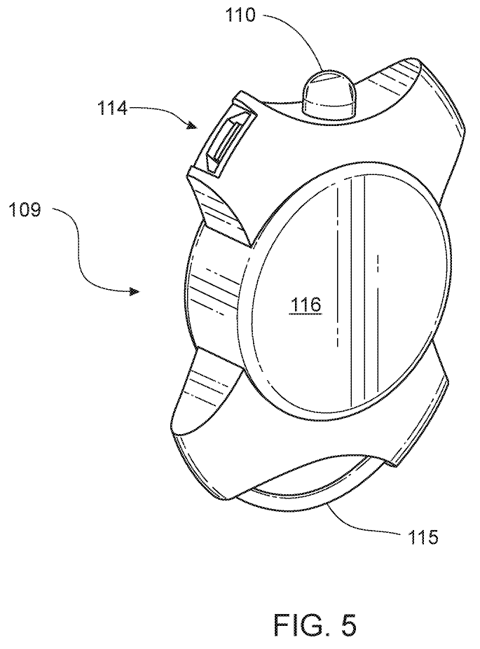

Still referring to FIG. 2, the second scissor component includes a plurality of measurement increments 111 above the blade 104B. In one embodiment, the plurality of measurement increments is from zero to five centimeters. In one embodiment, increments are provided at every half and full integer from zero to five centimeters. In one embodiment, the measurement increments are configured to measure wounds, distance, or any other measurement readings necessary. It should be understood that the measurement increments and units of measure can very without departing from the scope of the invention. In one embodiment, a removable light 109 is provided in the first scissor component. The removable light is removable from the first scissor component 101, and can be operable in the first handle component or removed from the first handle component, such that the removable light can be operated outside the scissors. In one embodiment, the body (116; FIG. 5) of the removable light acts as an on/off switch activating and deactivating a LED light 110. The removable light will be discussed in greater detail below.

Yet still referring to FIG. 2, in one embodiment, second handle component 106 includes a pocket clip 117 formed around the perimeter of second hole 108 adjacent to the first handle component. The pocket clip is useful for attaching the medical scissors to a pocket of a user, such that the bedside medical scissors can be transported hands-free. Similarly, a third hole 116 in the first handle component is provided. A key ring or other attachment can be used through the third hole as well known in the art. In one embodiment, a relief sink 118 is provided to prevent sink during manufacturing as well known in the art.

Referring now to FIG. 3, the back view of the bedside medical scissors is illustrated. It is a particular advantage of the present invention to provide a plurality of circles 112 etched into the back side of the first scissor component 101. In one embodiment, the plurality of circles range from two to six millimeters in diameter. In one embodiment, plurality of circles is configured to evaluate the size of skin lesions.

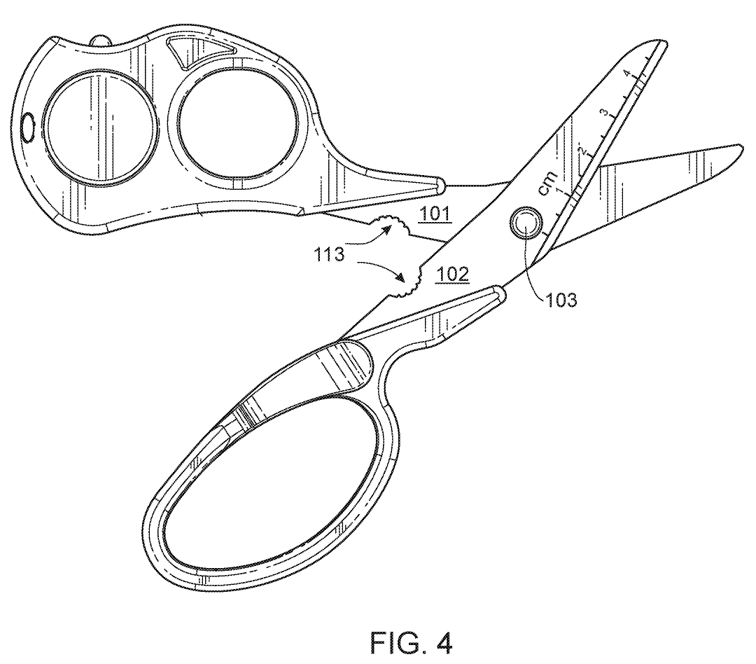

FIG. 4 is a front view of the bedside medical scissors in an open position according to an embodiment of the present invention. Referring now to FIG. 4, the bedside medical scissors are shown in an open position. From this view, the provided clamp 113 is visible, wherein each section of the clamp is included on the first scissor component 101 and the second scissor component 102 as illustrated. In one embodiment, the clamp is configured to attach to intravenous (IV) lines in order to easily remove the line from the IV bottle or IV bag as well known in the art. Often times, the IV lines and IV bags become stuck with adhering solutions, antibiotics, medications, blood, etc. The clamp provided helps the user clasp onto the lines or bags so they can be easily removed from each other. Likewise, the clamp can be used to remove other lines from various ports and medical devices well known in the art.

FIG. 5 is a perspective view of the removable light 109 removed from the bedside medical scissors according to an embodiment of the present invention. Referring now to FIG. 5, the removable light 109 is illustrated. As previously mentioned, the removable light includes a LED light 110 providing the user (such as a nurse) the ability to maneuver in a hospital room without turning on the lights, useful for night time shifts. In one embodiment, the LED light is particularly useful as an aid in inspecting a patient, as well as name bands, labels, and other uses where a direct light would benefit the user. In one embodiment, a connecting loop 115 is positioned on the opposite end from the LED light allowing the user to connect the removable light to a keychain, a lanyard, or the like. In one embodiment, the removable light is sized and shaped to fit into the first scissor component (as seen in FIGS. 1-4). The removable light is an interference fit with the first handle component 105, allowing for the removable light to be removed and installed into the bedside medical scissors easily by the user. In one embodiment, a micro USB port 114 is provided on the removable light such that the rechargeable battery (not illustrated) may be charged via a micro USB cable (not shown) when plugged into a power source as well known in the art. Conveniently, the USB port can be accessed when the removable light is installed in the first handle component. When the removable light is removed from the first handle component, the bedside medical scissors may be autoclaved to sterilize the scissors preventing the spread of infection between patients.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. Bedside medical scissors comprising:
a first scissor component including a first handle component, wherein the first handle component is formed to include a first hole configured to receive a thumb of a user;
a second scissor component including a second handle component, wherein the second handle component is formed to include a second hole configured to receive at least one finger of the user;
the first scissor component and the second scissor component each including a blade having a blade tip, wherein the first scissor component and the second scissor component are attached and configured to pivot via a pivot element forming a shear; and
a removable light having a light source, the removable light positioned in the first handle component, such that the light source is directed approximately perpendicular to the blades and away from the blade tip, wherein the removable light is operable while positioned in the first handle component and operable while removed from the first handle component of the bedside medical scissors.

2. The bedside medical scissors of claim 1, wherein the first scissor component includes a plurality of circles, each circle of the plurality of circles having a different sized diameter.

3. The bedside medical scissors of claim 2, wherein the plurality of circles range from two to six millimeters in diameter.

4. The bedside medical scissors of claim 1, wherein the second scissor component includes a plurality of measurement increments.

5. The bedside medical scissors of claim 4, wherein the plurality of measurement increments is from zero to five centimeters, and the plurality of measurement increments are located along the blade.

6. The bedside medical scissors of claim 1, wherein the first scissor component includes a first section of clamp and the second scissor component includes a second section of clamp, such that a clamp is provided.

7. The bedside medical scissors of claim 6, wherein the clamp is configured to attach to intravenous (IV) lines.

8. The bedside medical scissors of claim 1, wherein the bedside medical scissors are constructed of various materials which are configured to be autoclaved when the removable light is removed from the first handle component.

9. The bedside medical scissors of claim 1, wherein the removable light includes a micro USB port configured to enable the charging of a rechargeable battery.

10. The bedside medical scissors of claim 9, wherein the light source is an LED light powered by the rechargeable battery.

11. The bedside medical scissors of claim 1, wherein the removable light includes a connecting loop.

12. The bedside medical scissors of claim 1, further comprising a pocket clip positioned on the second handle component.

13. Bedside medical scissors comprising:
a first scissor component including a first handle component having a first inner surface and a first outer surface, wherein the first handle component is formed to include a first hole configured to receive a thumb of a user;
a second scissor component including a second handle component having a second inner surface and a second outer surface, wherein the second handle component is formed to include a second hole configured to receive at least one finger of the user;
the first scissor component and the second scissor component each including a blade having a blade tip, wherein the first scissor component and the second scissor component are attached and configured to pivot via a pivot element forming a shear, the first inner surface of the first handle component configured to abut the second inner surface of the second inner surface when the scissors are in a closed position; and
a removable light having a light source, the removable light positioned in the first handle component such that the light source is normal to the first outer surface, wherein the removable light is operable while positioned in the first handle component and operable while removed from the first handle component of the bedside medical scissors.
